(19) United States
(12) Patent Application Publication
(10) Pub. No.: US 2008/0149107 A1
(43) Pub. Date: Jun. 26, 2008

(54) Medico-Surgical Tubes

(76) Inventor: Joanna Byatt, Kent (GB)

Correspondence Address: Louis Woo;Law Office of Louis Woo, 717 N Fayette Street, Alexandria, VA, 22314, US

(21) Appl. No.: 11/883,891

(22) PCT Filed: Jan. 26, 2006
(86) PCT No.: PCT/GB2006/000268

§ 371 Date: Aug. 8, 2007

(30) Foreign Application Priority Data
Feb. 15, 2005 (GB) 0503081.2

(51) Int. Cl. A61M 16/04

(52) U.S. Cl. 128/207.14

(57) ABSTRACT

A tracheotomy tube has a movable flange (2) that can be locked where desired along the shaft (1) of the tube. The flange has two wings (24) and (25) hinged close to one end, which has a cam profile (28). When the wings are folded rearwardly the flange is movable freely along the shaft. The flange is locked in position by folding the wings and forwardly and outwardly so that the cams contact and fractionally grip the outside of the shaft. The wings are retained in the locking position by a tape threaded through apertures (26) in the outer ends (27) of the wings and by which the tube is secured with the patient's neck.

This invention relates to medico-surgical tubes of the kind comprising a tubular shaft and a flange mounted on and movable along the shaft, the flange including two wings arranged to extend laterally of the shaft for securing the tube with the patient's body.

Medico-surgical tubes, such as tracheostomy tubes, are commonly provided with a flange to secure the tube to the patient's body. In the case of a tracheostomy tube, the flange is positioned close to the surface of the neck where the tube enters the tracheostomy, a tape is threaded through openings in the flange and fastened around the neck. For most patients, a comfortable fit can be achieved using one of a range of several different size tubes, each having a flange mounted at a fixed location along the tube suitable for patients having an average anatomy. There are, however, some situations where a fixed flange is not suitable, such as, for example, in obese patients where tissue between the neck surface and the trachea is very thick. In these situations, it is preferable for the flange to be movable along the tube to the ideal position and to be lockable in that position. Tubes with adjustable flanges are described in, for example, U.S. Pat. No. 5,026,352, U.S. Pat. No. 4,249,529, U.S. Pat. No. 4,449,527, U.S. Pat. No. 4,498,903, U.S. Pat. No. 4,530,354, U.S. Pat. No. 4,530,354, U.S. Pat. No. 4,649,913, U.S. Pat. No. 4,683,882, U.S. Pat. No. 4,774,944, WO80/02645, WO84/03217 and U.S. Pat. No. 4,278,081. It is, however, difficult to achieve a secure fastening of the flange to the tube in a simple manner whilst also enabling the flange to be moved and secured easily, especially where the tube is wet and slippery.

It is an object of the present invention to provide an alternative medico-surgical tube.

According to one aspect of the present invention there is provided a medico-surgical tube of the above-specified kind, characterised in that at least one wing is angularly displaceable from a first position where the flange is free to move along the shaft to a second position where the wing extends substantially at right angles to the shaft for securing the tube with the patient's body and the wing prevents movement of the flange along the shaft.

The wing preferably extends towards the machine end of the shaft in the first position. Both wings are preferably displaceable from the first position to the second position. The or each wing is preferably mounted for angular movement about an axis extending across the width of the wing close to one end of the wing, the one end of the or each wing being arranged frictionally to contact the outside of the shaft to prevent movement of the flange relative to the shaft when the or each wing is in the second position. The one end of the or each wing may have a cam profile. The wings may have an aperture therein through which a tape can extend for use in securing the tube with the patient's body. The shaft may be arranged to extend through a tracheostomy so that a patient end of the shaft locates in the trachea, the flange being arranged to secure the tube with the patient's neck.

According to another aspect of the present invention there is provided a medico-surgical tube comprising a tubular shaft and a flange mounted on and movable along the shaft, the flange including two wings arranged to extend laterally of the shaft for securing the tube with the patient's body, characterised in that at least one wing has a first end with a cam formation and a second, outer end, and that the wing is angularly displaceable about an axis close to the first end of the wing from a first position where the flange is free to move along the shaft to a second position where the cam formation engages the shaft and prevents movement of the flange along the shaft.

Preferably each wing is provided with means for retaining the wing against the patient's body and thereby retaining the flange in a locked state with respect to the shaft.

According to a further aspect of the present invention there is provided a method of placing a tracheostomy tube of the kind having a movable flange with two retaining wings, including the steps of folding the wings to a first position where the flange is movable along a shaft of the tube, positioning the flange towards the machine end of the shaft, inserting the patient end of the shaft through a tracheostomy into the trachea, sliding the flange forwardly to abut the neck surface, folding the wings forwardly and outwardly to lock the flange in position on the shaft and to lie against the surface of the neck, and securing the wings with the neck to retain the flange in the locked position.

A tracheostomy tube according to the present invention will now be described, by way of example, with reference to the accompanying drawing, in which:

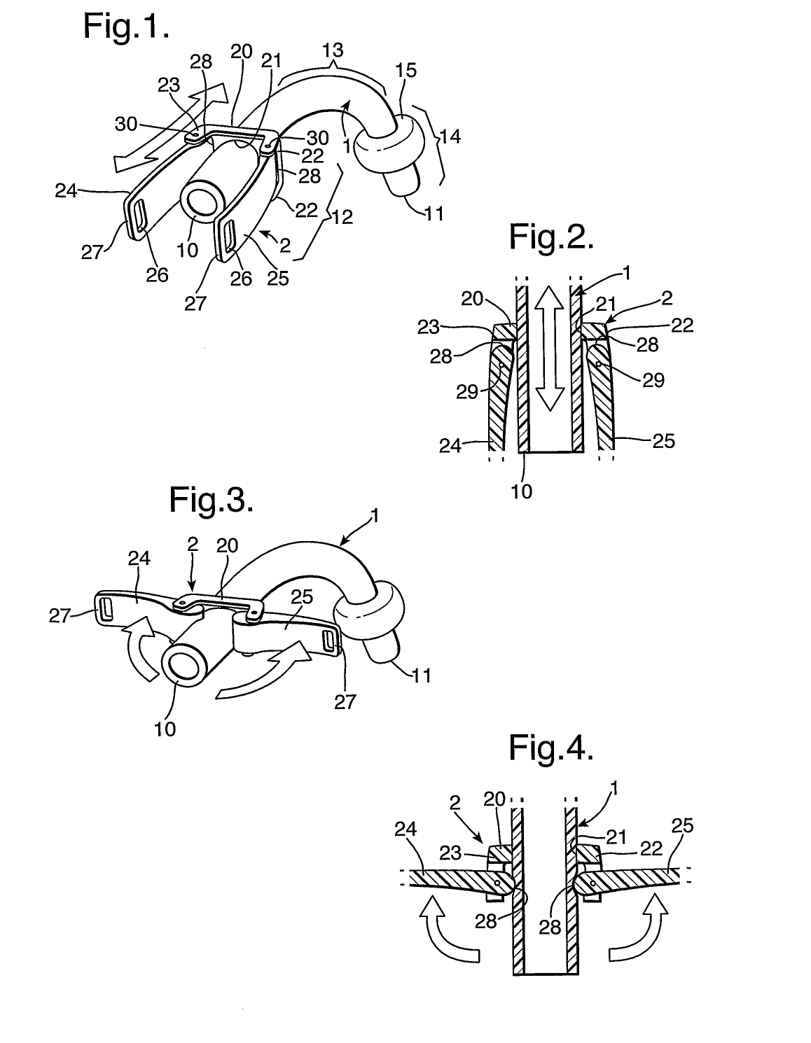

FIG. 1 is a perspective view of the tube with the flange in a released condition;

FIG. 2 is a sectional plan view of a part of the tube with the flange in a released condition;

FIG. 3 is a perspective view of the tube with the flange in a locked condition; and

FIG. 4 is a sectional plan view of a part of the tube with the flange in the locked condition.

The tube comprises a tubular shaft 1 and a flange 2, which is movable along the shaft and can be locked in any desired position along the shaft.

The shaft 1 is made of a conventional, bendable plastics material, such as PVC, polyurethane or silicone, is hollow with a circular section and has a smooth inner and outer surface. The shaft has a machine end 10 adapted to be located outside the body and may be connected to patient breathing apparatus or left open to air. At its opposite end, the shaft 1 has an open patient end 11 adapted to be located within the trachea and by which gas is supplied to and from the trachea. The shaft 1 comprises a straight machine end region 12, a curved intermediate region 13 and a straight patient end region 14 extending at substantially right angles to the patient end region. Alternative shape shafts are possible, such as shafts that are curved continuously along their length or shafts that have a natural straight shape but are highly flexible so that they can conform readily to the shape of the anatomy. Towards its patient end 11 the shaft supports a sealing cuff 15, which can be inflated and deflated via an inflation line (not shown) to seal the outside of the shaft 1 with the trachea when the patient end is correctly located in the trachea, so that gas is confined to flow along the inside of the shaft.

The flange 2 comprises a rectangular carriage plate 20 with a central circular aperture 21 the diameter of which is such that the shaft 1 is a close sliding fit within the aperture. Two pairs of mounting lugs 22 and 23 on opposite sides of the plate 20 extend rearwardly, that is, towards the machine end 10 of the shaft 1. Each pair of lugs 22 and 23 supports one end of a respective hinged wing 24 and 25 moulded from a rigid plastics material. Each wing 24 and 25 is rectangular and is formed with a lateral slot 26 towards its free end 27 by which a tape, strap or the like can be secured to the wing. At its other end, each wing 24 and 25 is formed with an enlarged, rounded cam formation 28 extending across the width of the wing. A steel mounting pin 29 extends through each wing 24 and 25 across its width in the region of the cam 28 and projects from the upper and lower edges of the wing a short distance rearwardly of the cam surface 28. The pins 29 locate in holes 30 in the lugs 22 and 23 so that the wings are angularly displaceable about parallel axes across the width of the wings extending at right angles to the axis of the shaft 1 and displaced laterally of the axis.

FIGS. 1 and 2 show the flange 2 in an unlocked condition with the wings 24 and 25 folded rearwardly, extending parallel to one another and to the axis of the shaft 1. In this position, the cam formations 28 are located away from the outer surface of the shaft 1 so do not impede movement of the flange 2 along the shaft 1. The flange 2 is set with the wings 24 and 25 in this first position during positioning of the tube in the patient. It is not essential that the wings be folded flat, parallel with the shaft, only that they extend generally towards the rear of the tube and could be at an angle. The flange 2 may be removed completely from the shaft 1, if desired, during insertion. When the shaft 1 has been correctly positioned, the flange 2 is moved close to the surface of the neck and is locked in position. To do this, the two wings 24 and 25 are folded outwardly, by pushing the free ends 27 of the wings outwardly and forwardly to a second position where they extend laterally, at right angles to the shaft and where they lie against the surface of the neck. As the wings 24 and 25 rotate about the axes of the pins 29, the cam formations 28 move into engagement with the outer surface of the shaft 1 and start applying a frictional gripping pressure to the shaft on opposite sides. The wings 24 and 25 are fully extended to the position shown in FIGS. 3 and 4 where they extend laterally outwardly at right angles to the axis of the shaft 1. In this position, the frictional, gripping force applied by the cam formations 28 is sufficient to prevent relative movement between the flange 2 and the shaft 1 so that the two are locked together. The wings 24 and 25 are prevented from moving rearwardly to the released condition because their outer ends 27 are secured by the tape used to attach the flange with the patient's neck.

The flange can be locked and unlocked readily by the user without the need for any special tools, whilst the force applied to the outside of the shaft can be relatively high because of the mechanical advantage achieved by virtue of the location of the displacement axes of the wings close to the cam end. Each wing can be locked and unlocked independently of the other. In this way, one wing can be unlocked and folded forward while the tube is in position in a patient so that the region of the stoma can be cleaned, treated or observed. The other wing remains locked so that the flange is still stabilised on the tube thereby inhibiting movement of the tube, which is known to be a problem with previous tubes. After the region under one wing has been cleaned it can be locked back in position and the other wing released and folded forwards to allow cleaning, treatment or observation of the rest of the region around the stoma.

The grip of the wings on the shaft can be improved in various different ways. For example, the gripping surface of the cam formations or the outer surface of the shaft towards its machine end, or both, could be provided with surface formations such as ribs, pips, knurling, roughening or the like. A layer of a high friction material, such as an elastomer, could be provided on the cam surfaces or on the machine end region of the shaft, or on both. This could be achieved by overmoulding the wings, shaft or both with the high friction material in the areas of contact.

Whilst it is preferable for both wings to be displaceable between a released and a locked condition, it is possible that a flange having one fixed wing and one displaceable, locking wing would be effective. The invention is not confined to tracheostomy tubes but could be used with other medico-surgical tubes having a flange for supporting the tube where it emerges from the body. It is not essential for the wings of the flange to be held with the body by straps extending through an aperture in the wings. Instead, the wings could be held by an adhesive layer on the side of the wings facing the body, by adhesive tape fastened over the wings and onto the skin, by sutures or by other means.

1. A medico-surgical tube comprising a tubular shaft and a flange mounted on and movable along the shaft, the flange including two wings arranged to extend laterally of the shaft for securing the tube with the patient's body, characterized in that at least one wing is angularly displaceable from a first position where the flange is free to move along the shaft to a second position where the wing extends substantially at right angles to the shaft for securing the tube with the patient's body and the wing prevents movement of the flange along the shaft.

2. A tube according to claim 1, characterized in that the wing extends towards the machine end of the shaft in the first position.

3. A tube according to claim 1, characterized in that both wings are displaceable from the first position to the second position.

4. A tube according to claim 1, characterized in that the or each wing is mounted for angular movement about an axis extending across the width of the wing close to one end of the wing, and that the one end of the or each wing is arranged frictionally to contact the outside of the shaft to prevent movement of the flange relative to the shaft when the or each wing is in the second position.

5. A tube according to claim 4, characterized in that the one end of the or each wing has a cam profile.

6. A tube according to claim 1, characterized in that the wings each have an aperture therein through which a tape can extend for use in securing the tube with the patient's body.

7. A tube according to claim 1, characterized in that the shaft is arranged to extend through a tracheostomy so that a patient end of the shaft locates in the trachea, and that the flange is arranged to secure the tube with the patient's neck.

8. A medico-surgical tube comprising a tubular shaft and a flange mounted on and movable along the shaft, the flange including two wings arranged to extend laterally of the shaft for securing the tube with the patient's body, characterized in that at least one wing has a first end with a cam formation and a second, outer end, and that the wing is angularly displaceable about an axis close to the first end of the wing from a first position where the flange is free to move along the shaft to a second position where the cam formation engages the shaft and prevents movement of the flange along the shaft.

9. A medico-surgical tube according to claim 8, characterized in that each wing is provided with means for retaining the wing against the patient's body and thereby retaining the flange in a locked state with respect to the shaft.

10. A method of placing a tracheostomy tube of the kind having a movable flange with two retaining wings, including the steps of folding the wings to a first position where the flange is movable along a shaft of the tube, positioning the flange towards the machine end of the shaft, inserting the patient end of the shaft through a tracheostomy into the trachea, sliding the flange forwardly to abut the neck surface, folding the wings forwardly and outwardly to lock the flange in position on the shaft and to lie against the surface of the neck, and securing the wings with the neck to retain the flange in the locked position.
